(19) United States
(12) Patent Application Publication
(10) Pub. No.: US 2002/0162141 A1
(43) Pub. Date: Oct. 31, 2002

(54) Transgenic plant protein expression and recovery system

(76) Inventors: T. Erik Mirkov, Harlingen, TX (US); James E. Irvine, South Padre Island, TX (US)

Correspondence Address: BAKER BOTTS L.L.P., 2001 ROSS AVENUE, SUITE 600, DALLAS, TX, 75201-2980, US

(21) Appl. No.: 10/119,992

(22) Filed: Apr. 10, 2002

Related U.S. Application Data
(63) Continuation-in-part of application No. 09/832,321, filed on Apr. 10, 2001.
(60) Provisional application No. 60/282,794, filed on Apr. 10, 2001.

(51) Int. Cl. A01H 5/00; A01H 1/00

(52) U.S. Cl. 800/288; 800/293

(57) ABSTRACT

A protein expression and recovery system in which transgenes are expressed in target plant varieties. Transformation is accomplished through bombardment of embryonic calli with plasmid-coated tungsten particles. Transformed calli are grown on tissue culture medium to produce transgenic plants which are then screened for presence of the transgene DNA or the polypeptide of interest. Transgenic plants are then cultivated. Cultivated plants may be harvested for extraction of the polypeptide.

### CLAIM OF PRIORITY

The present application claims priority to U.S. Provisional Application Serial No. 60/282,794 filed Apr. 10, 2001, which is incorporated by reference herein.

### GOVERNMENT RIGHTS IN THE INVENTION

This invention was made in part with United States Government support and the United States Government has certain rights in the invention.

### FIELD OF THE INVENTION

This invention relates to transgenic plants, particularly transgenic sugarcane, and the use thereof as a protein expression system.

### BACKGROUND OF THE INVENTION

Since the discovery several decades ago that recombinant animal proteins, such as insulin, may be produced in simpler life forms, the number of such recombinant or transgenic proteins has increased exponentially, as has demand. Traditional methods of protein production involve the expression of such proteins in bacteria or yeast followed by harvesting. However, such systems may produce improperly glycosylated or otherwise nonfunctional protein products, particularly if the protein is derived from a higher eukaryotic organism. Additionally, residual bacterial or yeast cell matter may cause serious immune responses or other problems if the proteins are later administered to humans or other animals.

One method of overcoming this problem involves production of a desired protein in insect or mammalian cells. This generally results in improved glycosylation, folding and other post-translational modifications as compared to bacterial and yeast systems. However, such systems must generally be maintained as tissue cultures grown in costly bioreactors. The expense of such systems has limited the application of recombinant or transgenic proteins. Additionally, bioreactors always pose the threat of contamination by bacteria, yeast or other fungi which may be harmful to patients or thwart other downstream uses of the protein. Purification systems designed to remove contaminant residues and tests for such residues, such as endotoxin, further increase the cost of proteins produced in such systems.

In order to avoid these problems, researchers have increasingly focused on transgenic plants as an expression system. Plants offer a number of advantages as compared to bacterial, yeast, insect cell, and mammalian cell expression systems. First, plants tend to properly post-transcriptionally modify many proteins, whether of plant or animal origin. Second, they can be grown cheaply outdoors or indoors with little control of conditions as compared to bioreactors. Third, certain plants tend to not produce extremely harmful cellular components and naturally keep many fungi and bacteria that produce such components under control. Fourth, many proteins expressed in plants do not even require purification because they may be benefit the plant directly, are consumed with the plant matter or are not destroyed by normal processing.

Genes may be introduced into plants in a variety of ways, including viral introduction and bombardment. See Gallo-Meagher, M. and Irvine, J. E. (1993), Effects of tissue type and promoter strength on transient GUS expression in sugarcane following particle bombardment. Plant Cell Reports 12:666-670, incorporated by reference herein. Selection may be accomplished using a variety of factors such as that described in Gallo-Meagher, M. and Irvine, J. E. (1995), Selecting sugarcane transformed with the bar gene, Prot. Int. Soc. Sugar Cane Tech. 22nd Congress, incorporated by reference herein.

A great deal of work in plants has focused on expression of transgenes in plants to confer protection upon that plant only. See Gallo-Meagher, M. and Irvine, J. E. (1995), Herbicide resistance in a transgenic sugarcane variety. Plant Physiology and U.S. Patent Ser. No. 5,850,025 to Mirkov et al. for “Protection of plants against plant pathogens”, both incorporated by reference herein.

Plants can be viewed as small efficient factories that need only water, sunlight, minerals, and the right combination of additional genes to economically produce exactly what industry wants. Given the right genes, plants can be used as recombinant expression systems to produce large quantities of modified starches, valuable industrial oils, plastics, pharmaceuticals, vaccines or enzymes for food processing and other industries.

Work in transgenic potatoes, bananas and rice which express transgenes has produced some proteins which may be consumed with the plant matter. Less research has been done in the area of proteins to be purified or processed. Additionally, because not every transgene of interest may be expressed in every plant, a variety of plant expression systems are required to allow expression of a range of proteins. Plants also vary in growth regions and conditions, thus a variety of systems is required to meet needs and allow development around the world. Finally, plants which grow quickly may provide a more efficient system than slower growing plants. Thus, additional plant transgenic expression systems are required to meet these and other protein production needs.

### SUMMARY OF THE INVENTION

The production of crop plants improved by the insertion of foreign genes constitutes one of the main goals of plant genetic engineering. The transgenic plant technologies of the present invention are useful for development and commercialization of molecular farming in transgenic grasses and other plants. The invention includes a method of producing a polypeptide or protein in a transgenic plant. First a target plant variety and transgene DNA molecule encoding the polypeptide are selected. A plasmid is constructed which includes a ubiquitin promoter or other promoter active in the target plant variety and a nopaline synthase transcriptional terminator or other transcriptional terminator operably linked to the transgene DNA molecule. Embryonic calli of the target plant variety may then be transformed using tungsten particles coated with the plasmid. The bombarded calli may then be cultured on a tissue culture medium to regenerate transgenic plants. The transgenic plants may be cultivated and the polypeptide later extracted from the cultivated plants.

Any polypeptide, including proteins may be thus produced. In an exemplary embodiment the protein is a plant or mammalian protein. In more specific embodiments these proteins may be snow drop lectin, BAR, bovine lysozyme, Peptidyl MTM, or SCMV-H coat protein.

The target plant variety may be from any plant family, but in an exemplary embodiment is a sugarcane variety. Particularly, the plant variety may be CP65-357 or CP72-1210.

The tungsten particles which bombard the embryonic calli may be propelled from any conventional source, such as, but not limited to a helium driven particle inflow gun. Each tungsten particle may contain approximately 4 μg of the transgene DNA molecule.

To allow selection of transformed plants, the calli may also be transformed with a DNA sequence encoding a selectable marker protein. The DNA sequence may be included in the plasmid. It may also be part of a construct with which the calli are co-transformed. One suitable construct comprising a selectable marker is the maize ubiquitin/nptII gene construct.

If a selectable marker has been introduced, the calli and resulting transgenic plants may be cultivated in a selective tissue culture medium. The tissue culture medium may be supplemented to promote shoot generation.

The resulting transgenic plants may be screened for the presence of the transgene DNA molecule or the polypeptide. Such screening may include Western and Southern blots, PCR and other detecting techniques.

Plants which contain the transgene may then be cultivated and later harvested. Plant matter containing the polypeptide may then be collected. In many plants, collection may occur by crushing the plant to obtain juice which contains the polypeptide. The juice or other liquid medium containing the protein may then be sent to an extraction process. Methods of extracting the polypeptide include filtration, HPLC, ion exchange resin extraction or hydrophobic interaction resin extraction. Certain plants will benefit directly from the polypeptide and thus no extraction will be necessary.

The above summary provides a general outline of the invention. For a better understanding of the invention and its advantages, reference may be made to the following description of exemplary embodiments.

### DETAILED DESCRIPTION OF THE PREFERRED EMBODIMENTS

As embodied and broadly described herein, the present invention is directed to a transgenic plant protein expression system. It also includes the processing of transgenic plants such as sugarcane for the recovery of high value proteins.

The invention also relates to methods of genetic engineering and manufacturing of products, such as high value proteins, from transformed plants such as sugarcane. The invention also includes products, such as proteins, produced according to the invention. The invention is not limited to sugarcane, but may be applied to other plants, such as sorghum.

Because of high biomass potential, multi-functional utility, existing processing plants and other features, sugarcane has been investigated for use in the recombinant expression system of the present invention. In addition to the fact that sugarcane produces more biomass per acre than any other annual crop, the following unique features make sugarcane a particularly useful recombinant expression system. First, the per weight basis of protein in the extracted sugarcane juice is 0.2% and the remainder is mostly sucrose and water. The sucrose provides stabilization of the heterologous protein. Second, because the overall protein content is low, the starting material for purifying the recombinant protein is a simple mixture which facilitates the purification process.

The heterologous genes expressed in sugarcane according to one embodiment of the present invention produce bactericidal lytic peptides and proteins or insecticidal and antiviral lectins that have high value as antimicrobial pharmaceuticals or biopesticides. The large biomass produced by these crops, and the milling technology in place for sugarcane offers an increased capacity to capture transgenically expressed proteins as value added products as compared to other plant expression systems. Transgenic peptides and proteins in sugarcane are often found in the normally discarded residue of the first processing step after milling, juice clarification. The large scale extraction and purification of these value added products from the normally discarded residue generated in the first step of the sugarcane milling process, and commercialization of this technology will provide greater and cheaper access to the products. Furthermore, by combining the strengths of classical crop improvement and plant biotechnology, the invention provides avenues for crop gene manipulation for crop improvement. The transformation and extraction technology of the invention may be applied to a broad range of proteins and other plants, such as sorghum.

There are two main bottlenecks for improving crop plants by gene transfer. First, many useful genes have not been precisely identified. The second major bottleneck concerns the inability to regenerate plants from the cells into which the new genes have been transferred. These constraints have been overcome according to the invention. The invention provides a reproducible biolistic based transformation and regeneration system for creating transgenic disease and herbicide resistant sugarcane. Using the invention, sugarcane can be successfully transformed on a routine basis.

In one embodiment, cDNA coding for the snow drop lectin, a potent broad spectrum insecticidal and antiviral protein found in the bulb of the snow drop lily is transgenically expressed in sugarcane. This protein may have many potential uses in the biopesticide industry.

Using standard molecular biology techniques, the gene encoding the snow drop lectin may be fused between the maize ubiquitin promoter (a strong constitutive promoter in grasses) and the nopaline synthase transcriptional terminator in a high copy number plasmid. This construct may then be used in biolistic co-transformation, for example using the maize ubiquitin/nptII gene construct (resistance to the antibiotic geneticin) as the selectable marker. In an exemplary embodiment, the initial sugarcane cultivar to be transformed may be CP65-357, which is easy to regenerate. Targets of embryogenic calli may be produced by culturing immature flower inflorescence on tissue culture medium supplemented with 3 mg/l 2,4-D. These embryogenic calli may then be bombarded (using a helium driven particle inflow gun) with tungsten particles coated with the appropriate plasmid DNA molecules to provide 4 ug per shot. The plants may then be cultured on tissue culture medium supplemented with 3 mg/l 2,4-D and 45 mg/l geneticin. After 8 weeks, resistant calli may be transferred to medium supplemented with 1 mg/l 2,4-D and 45 mg/l geneticin to promote shoot regeneration. In one embodiment, shoots are subcultured every two weeks on this medium for two months, at which time they are placed on rooting medium containing 45 mg/l geneticin. Plants displaying well developed roots may be screened for the presence and expression of the transgene by PCR, Southern and Western blot analyses. In an exemplary embodiment, a set of the highest expressors may be grown in the greenhouse, and then in the field. The transgenically expressed protein may then be purified from these transgenic plants using tissue extraction, differential ultrafiltration, and ion exchange HPLC, or other extraction methods.

Although varieties which easily regenerate may provide a good target for the generation of transgenic plants according to the present invention, it will be understood that other plant varieties will be suitable so long as they can be induced to regenerate. Certain other factors, such as ease of transformation, lack of large natural protein quantities, absence of proteins similar to the transgenic proteins, desirable growth range and conditions and other factors may influence selection of the particular plant variety used to express any given transgene or transgenes according to the present invention.

The invention also encompasses a nondestructive methods for recovering high value proteins or peptides (e.g., pharmaceutical peptides) from transgenic sugarcane or other transgenic plants produced according to the present invention. In normal cane processing, the industry crushes the stalk to extract the juice, then washes the residue with water to complete the extraction. This mixture is then adjusted to approximately pH 7.0 with lime and heated to around 90° C. Then the flocculent is removed and the juice evaporated to syrup for crystallization of the sucrose. New technology for juice clarification developed in beet processing is now being adapted for cane processing. This technology enables the raw process material to be clarified without heating or liming. The process involves micron filtration to remove high molecular weight materials from the juice and leaves a clear filtrate that contains the high value proteins. According to one embodiment of the present invention, this fraction may then be used for protein extraction and purification. Extraction may be accomplished using a micron filtration unit coupled to ultramicron filtration and ion exchange chromatography units which separate the protein fraction of the juice and prepare it for further purification.

In an exemplary embodiment of the purification process, the transgenic sugarcane is first shredded and crushed twice (without maceration water). Essentially, the cane stalk is shredded and then pressed through 3 rollers on a Squire mill with around 3,000 pounds per square inch pressure. This produces a mixture of about 70% water, 15% sucrose, and 10% fiber. The remaining 5% of the mixture includes proteins and other sugars, salts and organic molecules. The juice containing the high value protein is then pumped to a purification skid and filtered through a set of vibrating (self cleaning) screens and enters Tank 1. This step substantially removes the fiber. The first screen is preferably 150 microns, and the second is preferably 100 microns. The pH of the juice is adjusted to around 5.2 and it is supplemented with approximately 1 mM EDTA and 0.1% sodium sulfite to prevent oxidation and the formation of phenolics. From Tank 1, the juice is permeated through a 0.2 micron cross flow filtration membrane. This step removes substantially all of the insoluble solids and high molecular weight soluble solids such as bacteria, starches and dextrans. The permeate, which contains sugar and the high value protein, enters Tank 2 and the retentate in Tank 1 may be discarded. From Tank 2, the juice is permeated through a 0.05 micron membrane. This step removes soluble molecules with a molecular weigh greater than 150,000 kd. High value proteins with a molecular weight greater than 150,000 kd. would be retained in Tank 2, and could be further purified with the HPLC steps described below. The second permeate, which contains sugar and the high value proteins smaller than 150,000 kd (snow drop lectin in this example) enters Tank 3 and the retentate in Tank 2 is discarded. At this point a relatively clean sample is present from which substantially all high molecular weight material has been removed, i.e., bacteria, starch, dextrans, and proteins with high molecular weights.

From Tank 3, the sample is further purified by 2 cycles of high pressure liquid chromatography (HPLC). The first cycle uses Dowex Mono 66 ion exchange resin, while the second cycle uses a hydrophobic interaction resin. Further modifications can be made to address large volumes produced in Tank 3. The first two membranes process the juice at 2 gallons per minute, but the HPLC can only handle 300 mL per minute. To assist with this problem, low molecular weight cut off membranes that can be used to concentrate the sample in Tank 3. The water, sugars and other small molecules will flow through the membranes, but the high value protein will be concentrated in Tank 3. This may improve the performance of the HPLC steps. Further modifications using different initial extraction conditions, different ion exchange resins/membranes, affinity resins and HPLC columns can be used to enhance performance. The exact equipment to be used will depend somewhat upon the size of the protein to be purified and its other properties as well as properties of the target plant and its native proteins.

Additional useful instrumentation which may be incorporated include pilot scale nano filtration (30,000 and 10,000 molecular weight cut-off) equipment, new HPLC columns and new ion exchange resins/membranes. Processing plants employing the extraction techniques according to the invention described herein, or incorporating an ultramicron filtration unit coupled to a de-watering system, may be used to extract and purify transgenically expressed proteins, including such biologically active high value proteins as pharmaceutical proteins, biopesticides, and lytic peptides.

The invention allows for the rapid economic production of large quantities of high value proteins. Transgenic plants may be produced resulting in large amounts of transgenic plant material that can rapidly be processed to produce large quantities of recombinantly expressed proteins. Slight modifications to the initial extraction may be made for different types of starting materials, and the size exclusion of the molecular weight cut off membranes could be altered for each specific protein, as could be the final HPLC steps.

The following additional examples are offered to illustrate embodiments of the invention, and should not be viewed as limiting the scope of the invention.

### EXAMPLES

### Example 1-Development of Transgenic Grasses for Molecular Farming

This example relates to developing transgenic grasses suitable for molecular farming. Because of high biomass potential and multi-functional utility, sugarcane or sorghum may be used. The first step is introduction of genes into these crops which will economically produce high value lytic peptides and proteins to be used in the pharmaceutical and biopesticide industries. Sugarcane and sorghum, closely related plants, are very efficient producers of biomass, and the sugarcane milling process is an efficient biomass extraction system. Transgenically expressed peptides and proteins would be expected in the normally discarded residue of the first processing step after milling, juice clarification.

Genetic transformation of grass-like crops has previously been slow because the methods of gene transfer that work for broadleaf plants are not suitable. The present invention includes a particle bombardment transformation system and a regeneration and screening technique which may be used to produce transgenic sugarcane that is herbicide resistant. Significant progress has been made in applying the technique to varieties of sorghum. For example, in connection with herbicide resistance and enhanced disease control, using a helium gun, sugarcane has been transformed with a UBI-bar construct and selected for resistance to bialophos.

Using a herbicide resistance gene as a selectable marker for transformed plants, embryogenic callus from sugarcane and sorghum were bombarded with plasmid DNA containing sequences coding for lytic peptides. Expression of these cDNAs linked to the maize ubiquitin promoter were assayed in transgenic plants by Northern and Western blots. Peptide activity was estimated by tissue extraction, dialysis and bioassays. Transgenic plants were then field grown for processing.

### Example 2-Molecular Farming with Transgenic Gramineous Crops

In this example, sugarcane and sorghum are also used to express lytic peptides and proteins that have high value as pharmaceuticals or biopesticides. As noted, the invention includes a reproducible biolistic based transformation and regeneration system for creating transgenic herbicide resistant sugarcane and sorghum. This system is adaptable to other plants and may be used, inter alia, to insert cDNA molecules that code for lytic peptides or proteins that have high value as pharmaceuticals or biopesticides.

Specifically, this example relates to transgenically expressing in sugarcane and sorghum the cDNA coding for bovine lysozyme, a potent broad spectrum bactericidal protein found in cow rennin. See U.S. Pat. No. 5,850,025. This protein has many potential uses in the biopesticide industry. For example, the purified protein is extremely effective in decontaminating bacterial infested seed, is an effective topical agent for both prophylactic and curative uses. Additionally transgenic plants expressing bovine lysozyme are resistant to bacterial infection. The cDNA for bovine lysozyme has been successfully expressed in tobacco, potato, tomato, and rice. These plants as well as sugarcane and sorghum can likely also express cDNA coding for the Peptidyl MIM™ DEM C-1, obtained from Demeter Biotechnologies, Ltd. This bio-compound is an effective antimicrobial against plant and animal diseases.

The volume and price at which these kinds of therapeutic proteins can be produced will determine to what extent they will be available for use. Current production methods are too expensive to allow for widespread market penetration. The cost of production of Peptidyl MIMs™ can be as much as $10,000 per gram when produced synthetically, and bovine lysozyme has not been synthesized. In recombinant yeast expression systems, the cost of production ranges from a low of $2.00 per gram for certain Peptidyl MIMs™ to $1,000 per gram for bovine lysozyme. Based on current expression levels of heterologous proteins being achieved in transgenic plants, the methods of the present invention allow to production of these proteins for as low as 05 cents per gram using sugarcane and sorghum as recombinant expression systems.

Using standard molecular biology techniques, cDNA encoding the bovine lysozyme peptide and the DNA molecule encoding the Peptidyl MIM™ DEM C-1 were fused between the maize ubiquitin promoter (a strong constitutive promoter in the Gramineae) and the nopaline synthase transcriptional terminator in a high copy number plasmid. This construct was used in biolistic co-transformation experiments using the maize ubiquitin/bar gene construct (resistance to the herbicides Ignite and Herbeace) as the selectable marker (1, 2, and 3). The initial sugarcane cultivar to be transformed was CP70-321, selected because it is the most widely grown cultivar in Texas, and is easy to regenerate. The grain sorghum variety Pioneer 8313 was used initially because embryogenic calli may be generated from floral meristems and plants have been reengineered from this tissue, and it is widely grown in south Texas. Targets of embryogenic calli were produced by culturing immature flower inflorescences on MS medium supplemented with 3 mg/l 2,4-D (3). These embryogenic calli were bombarded (using a helium driven particle inflow gun) with tungsten particles coated with the appropriate plasmid DNAs to provide 4 pg per shot. Plants were cultured on MS medium supplemented with 3 mg/l 2,4-D and 5 mg/l Ignite. After four weeks, Ignite resistant calli will be transferred to MS medium supplemented with 1 mg/l 2,4-D and 5 mg/l Ignite to promote shoot regeneration. Shoots were subcultured every two weeks on this medium for two months at which time they were placed on rooting medium containing Ignite. Plants displaying well developed roots were screened for the presence and expression of the transgene by PCR and Western blot analyses. A set of the highest expressors were grown in the greenhouse. The transgenically expressed proteins were partially purified from these transgenic plants using tissue extraction, dialysis, and differential ultrafiltration. The protein activity was bioassayed using several species of plant pathogenic bacteria for generation of kill curves. Further purification and bioassays were carried out. Small plots of these transgenic plants were then be field grown for initial pilot plant processing experiments to be conducted using a pilot plant located at the sugar mill in Santa Rosa, Tex.

Transgenically expressed peptides and were found in the normally discarded residue of the first processing step after milling, juice clarification. This juice was used as the starting material for partial purification and bioassays as described above.

### Example 3-Engineering Resistance to Sugarcane Mosaic Virus

Sugarcane mosaic virus (SCMV) and sorghum mosaic virus (SrMV) are aphid transmitted potyviruses with single stranded RNA genomes. There are several strains that cause significant losses in sugarcane growing areas throughout the world. These viruses have been difficult to control in cultivated varieties by the transfer of virus resistance genes from naturally resistant varieties through traditional breeding programs. However, it has now been demonstrated that it is possible to control potyviruses very effectively by genetic engineering. This technique is known as “coat protein-mediated resistance” and is a form of pathogen derived resistance. It has been demonstrated for many viruses, and in many plants, that the virus may be controlled by transforming the plant with the virus gene that produces its coat protein. However, no such transformative protection of sugarcane has yet been achieved.

According to the method of the present invention, transgenic sugarcane plants that express the coat protein of SrMV stain H may be produced which are resistant to this and other closely related strains of SCMV. This engineered resistance is monogenic and, therefore, easily transferred to other sugarcane varieties by conventional plant breeding methods. The resistance gene may be transferred using a plasmid containing nucleic acid encoding the SCMV-H coat protein and a UBI-bar selectable marker construct. In particular, resistant versions of the Nco 310 and CP72-1210 varieties may be produced and the resistance gene may be transferred though traditional breeding.

Other embodiments and uses of the invention will be apparent to those skilled in the art from consideration of the specification and practice of the invention disclosed herein. It is intended that the specification and examples be considered exemplary only.

We claim:

1. A method of producing a polypeptide comprising:
selecting a target plant variety;
selecting a transgene DNA molecule encoding the polypeptide;
constructing a plasmid comprising a ubiquitin promoter and a nopaline synthase transcriptional terminator operably linked to the transgene DNA molecule;
transforming an embryonic calli of the target plant variety with tungsten particles coated with the plasmid;
culturing the bombarded calli on a tissue culture medium to regenerate transgenic plants;
cultivating the transgenic plants.

2. The method of claim 1 wherein the polypeptide is a protein.

3. The method of claim 2, wherein the protein is snow drop lectin.

4. The method of claim 2, wherein the protein is BAR.

5. The method of claim 2, wherein the protein is bovine lysozyme.

6. The method of claim 2, wherein the protein is Peptidyl MTM.

7. The method of claim 2, wherein the protein is the SCMV-H coat protein.

8. The method of claim 1, wherein the target plant variety is selected from the families consisting of sugarcane and sorghum.

9. The method of claim 1, wherein the target plant variety is sugarcane variety CP72-1210.

10. The method of claim 1, wherein the target plant variety is CP65-357.

11. The method of claim 1, wherein the each tungsten particle is coated with approximately 4 μg of transgene DNA molecule.

12. The method of claim 1, wherein the calli are additionally transformed with a DNA sequence encoding a selectable marker protein.

13. The method of claim 12, wherein the DNA sequence encoding a selectable marker protein is included in the plasmid.

14. The method of claim 12, wherein the transformed calli are co-transformed with a construct comprising the DNA sequence encoding a selectable marker protein.

15. The method of claim 14, wherein the construct comprising a selectable marker is the maize ubiquitin/nptII gene construct.

16. The method of claim 1, in which transgenic plants are screened for the presence of the transgene DNA molecule.

17. The method of claim 1, in which the transgenic plants are screened for the presence of the polypeptide.

18. The method of claim 1, additionally comprising the step of extracting the polypeptide from the cultivated plants.

19. The method of claim 18, wherein the polypeptide is extracted by crushing the plants to collect juice.

20. The method of claim 19, wherein the polypeptide is extracted from the juice using at least one of the following techniques: filtration, HPLC, ion exchange resin extraction or hydrophobic interaction resin extraction.
